(19) United States
(12) Patent Application Publication
(10) Pub. No.: US 2025/0136622 A1
(43) Pub. Date: May 1, 2025

(54) CHIRAL N-HETEROCYCLIC CARBENE LIGAND-PROTECTED COPPER CLUSTER

(71) Applicant: Zhengzhou University, Zhengzhou (CN)

(72) Inventors: Shuangquan ZANG, Zhengzhou (CN); Xiyan DONG, Zhengzhou (CN); Xiaohong MA, Zhengzhou (CN)

(21) Appl. No.: 18/951,579

(22) Filed: Nov. 18, 2024

Related U.S. Application Data
(63) Continuation-in-part of application No. PCT/CN2024/097901, filed on Jun. 7, 2024.

(30) Foreign Application Priority Data
Oct. 9, 2023 (CN) 202311298297.X

(51) Int. Cl. C07F 1/08; C07D 401/14

(52) U.S. Cl. CPC C07F 1/08; C07D 401/14

(57) ABSTRACT

A chiral N-heterocyclic carbene ligand-protected copper cluster, having a formula of C₁₇₇H_{166.5}Cu₆F₃₆N_{31.5}P₆, abbreviated as R/S—Cu₃, belonging to a monoclinic crystal system and having a chiral space group P2₁, R—Cu₃: a=13.5254(2) Å, b=51.4781(5) Å, c=14.9912(2) Å, α=90°, β=116.3120(10), γ=90°, V=9356.4(2) Å³; S—Cu₃: a=13.5669(2) Å, b=51.5432(6) Å, c=15.0017(2) Å, α=90°, β=116.211(2), γ=90°, V=9411.7(3) Å³.

### CROSS-REFERENCE TO RELATED APPLICATIONS

This application is a continuation-in-part of International Patent Application No. PCT/CN2024/097901 with an international filing date of Jun. 7, 2024, designating the United States, now pending, further claims foreign priority benefits to Chinese Patent Application No. 202311298297.X filed Oct. 9, 2023. The contents of all of the aforementioned applications, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 245 First Street, 18th Floor, Cambridge, MA 02142.

### BACKGROUND

The disclosure relates to an interdisciplinary field of coordination chemistry and nanomaterials, and more particularly to an efficient pure blue light copper cluster protected by chiral N-heterocyclic carbene.

Atomically precise coinage metal (gold, silver, copper) clusters are a class of clusters with a precise atomic structure, and include an outer layer protected by an organic ligand and an inner core including three or more coinage metal atoms. Nanoclusters are a state of matter between atoms, molecules and bulk materials, and are a bridge connecting atoms, molecules and macroscopic matter. The sizes of the clusters are generally on the order of nanometers, thus presenting quantum confinement effect that gives rise to many new phenomena and properties, and producing excellent photophysical properties.

The excellent optical properties, light resistance, and good biocompatibility of nanoclusters make them have a wide range of applications. Currently, the research on coinage metal nanoclusters mainly focuses on the synthesis and application of novel nanoclusters, and the reported room-temperature luminescence of coin metal nanoclusters is mainly in the long wavelength region (e.g., yellow, red, etc.), and the luminescence quantum efficiency is relatively low, and these defects have greatly limited the application and development of nanoclusters.

Chirality is a universal phenomenon in nature and the chirality of nanoclusters can be conferred by chiral ligands. Chiral nanoclusters can be synthesized by choosing suitable chiral ligands. The excellent luminescent properties of chiral nanoclusters have important applications in circularly polarized organic light-emitting diodes, information storage, 3D displays, etc.

### SUMMARY

One objective of the disclosure is to provide a highly efficient pure blue copper cluster protected by chiral N-heterocyclic carbine ligand.

To achieve the objective, the disclosure provides a chiral N-heterocyclic carbene ligand-protected copper cluster, having a formula of C₁₇₇H_{166.5}Cu₆F₃₆N_{31.5}P₆, abbreviated as R/S—Cu₃, belonging to a monoclinic crystal system and having a chiral space group P2₁, R—Cu₃: a=13.5254(2) Å, b=51.4781(5) Å, c=14.9912(2) Å, α=90°, β=116.3120(10), γ=90°, V=9356.4(2) Å3; S—Cu₃: a=13.5669(2) Å, b=51.5432(6) Å, c=15.0017(2) Å, α=90°, β=116.211(2), γ=90°, V=9411.7(3) Å³;
the chiral N-heterocyclic carbene ligand having a formula as follows:

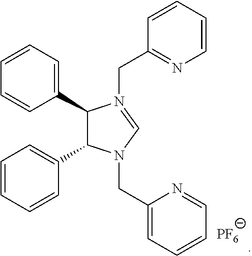

The chiral carbene ligand of the disclosure is prepared as follows:

A mixture of (1R,2R)-(+)-1,2-diphenyl-1,2-ethanediamine or (1S,2S)-(−)-1,2-diphenyl-1,2-ethanediamine and glyoxalate monohydrate in methanol solution was stirred at room temperature. N-bromosuccinimide (NBS) was added to the mixture and stirred at room temperature overnight. The reaction was quenched by the addition of saturated sodium metabisulfite and the solvent was removed in vacuum to obtain a solid, which was extracted with ethyl acetate by the addition of NaOH solution. The organic layer was dried with anhydrous magnesium sulfate and the solvent was removed in vacuum to obtain chiral imidazole. The synthesized chiral imidazole was dissolved in ethanol, and 2-(chloromethyl)pyridine hydrochloride and sodium bicarbonate were added and refluxed for two days. After filtration, the solvent was removed in vacuum, and a solid was collected, dissolved in dichloromethane, dried with anhydrous magnesium sulfate, and filtered. The solvent was removed in vacuum to obtain an oily product which was then mixed with tetrahydrofuran to obtain a powder. The powder was further washed with tetrahydrofuran and dried in vacuum to obtain a white powder. In a flask, the white powder was dissolved in methanol and excess aqueous ammonium hexafluorophosphate solution was dropped, whereby a precipitate was produced immediately. The precipitate was filtered, washed thoroughly with water/methanol and dried in vacuum to obtain the R/S—NHC^{py}-H·PF₆ chiral carbene ligand (as shown in FIG. 1).

The chiral pure blue copper cluster is prepared as follows:
the obtained chiral ligand was stirred with excess copper powder in acetonitrile. The suspension was filtered and the volume was reduced, and ether was added to obtain a white crude powdery product. The white crude powdery product was dissolved in acetonitrile, and colorless crystals were obtained through gas diffusion with ether, filtered, washed with ether and dried at room temperature.

The chiral pure blue copper cluster comprises three copper metal atoms, three organic ligands and three free hexafluorophosphate anions in a periphery of the three copper metal atoms; wherein the three copper metal atoms form a regular triangular framework having a flat surface; imidazole C atoms of chiral N-heterocyclic carbene ligand and a Cu₃ backbone surface form a coplanar C—Cu—C—Cu—C—Cu six-membered ring; the six-membered ring comprises three C—Cu—Cu isosceles triangles, which share one edge with the regular triangular framework of Cu₃; and each Cu atom is coordinated with two pyridines of two adjacent N-heterocyclic carbene ligands.

The chiral N-heterocyclic carbene-protected high-efficiency pure blue copper cluster of the disclosure features properties described as follows:

The copper cluster material of the disclosure has an intense pure blue phosphorescence at room temperature, and the emission spectrum thereof almost overlaps with that of commercial blue phosphors; its optimal emission wavelength is 456 nm (excitation wavelength 370 nm) (shown in FIG. 3); the CIE chromaticity coordinates are (0.014, 0.10) (shown in FIG. 4); and its maximum luminescence quantum yield is as high as 70% in thin film state, which is rare among the already reported N-heterocyclic carbene ligand-protected blue clusters. Higher quantum yield is a necessary condition for excellent luminescent materials, and high quantum yield blue light materials are the goal pursued by cluster researchers. The chiral ligand endows the entire cluster molecule with chiral properties (shown in FIG. 5), and the chiral structure as well as the strong luminescence give the material strong chiral luminescence properties (shown in FIG. 6). The strong chiral optical activity and solubility of the cluster at room temperature make the material very promising for application as an ink component in 3D printing for macroscopic models, such as the printing of metal cluster models with circularly polarized luminescent activity having precise atomic size (shown in FIGS. 7, 8) and for the printing of white “rabbit” model (FIGS. 9, 10) in combination with a chiral yellow light material (Ag₆L₆/Ag₆D₆). The use of the model for optoelectronic devices demonstrates the potential application of circularly polarized luminescent cluster materials in optoelectronic devices.

The following advantages are associated with the chiral N-heterocyclic carbene ligand-protected copper cluster of the disclosure. The chiral copper cluster materials of the disclosure are a class of rare blue luminescent materials, have pure blue luminescence that is basically the same as that of commercial blue phosphors at room temperature, with the optimal emission wavelength of 456 nm, and the quantum yield as high as 70%, which is an ultra-high luminescence quantum yield. The chiral ligands give the whole cluster molecules with chiral properties. The chiral structure as well as the strong luminescence gives the copper cluster a strong chiral luminescence property.

### BRIEF DESCRIPTION OF THE DRAWINGS

FIG. 1 shows a molecular structural diagram of a chiral carbene ligand of the disclosure;

FIG. 2 shows a schematic diagram of a pair of enantiomers of a chiral copper cluster material of the disclosure;

FIG. 3 shows excitation-emission curves of a chiral copper cluster material of the disclosure;

FIG. 4 is a graph of the emission spectra and chromaticity coordinates of a chiral copper cluster material of the disclosure and a commercial blue light phosphor;

FIG. 5 shows a circular dichroism plot of a chiral copper cluster material of the disclosure;

FIG. 6 shows a circularly polarized luminescence profile of a chiral copper cluster material of the disclosure;

FIG. 7 shows a macroscopic cluster model prepared by 3D printing with a chiral copper cluster material of the disclosure as an ink component;

FIG. 8 shows a circularly polarized luminescence graph of a macroscopic cluster model printed with a chiral copper cluster material of the disclosure as an ink component;

FIG. 9 shows a “rabbit” model printed with a chiral copper cluster material of the disclosure in combination with a yellow light material as an ink component; and

FIG. 10 shows a circularly polarized luminescence diagram of a “rabbit” model printed with a chiral copper cluster material of the disclosure as an ink component.

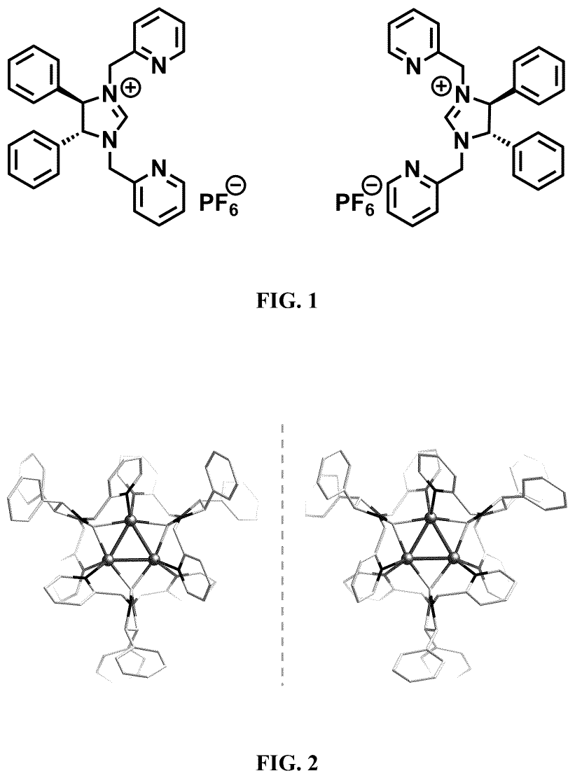

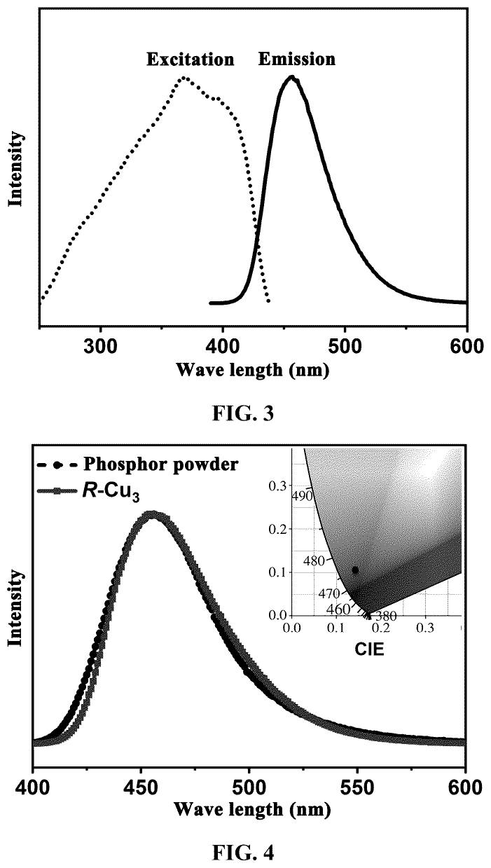

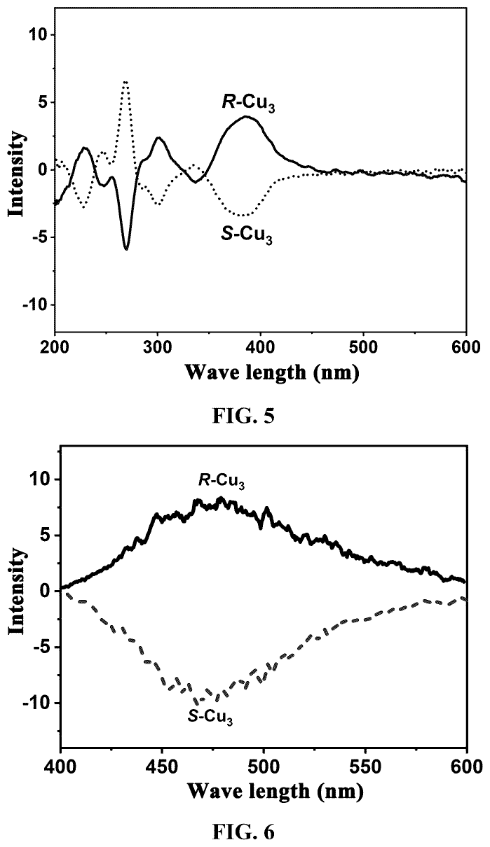

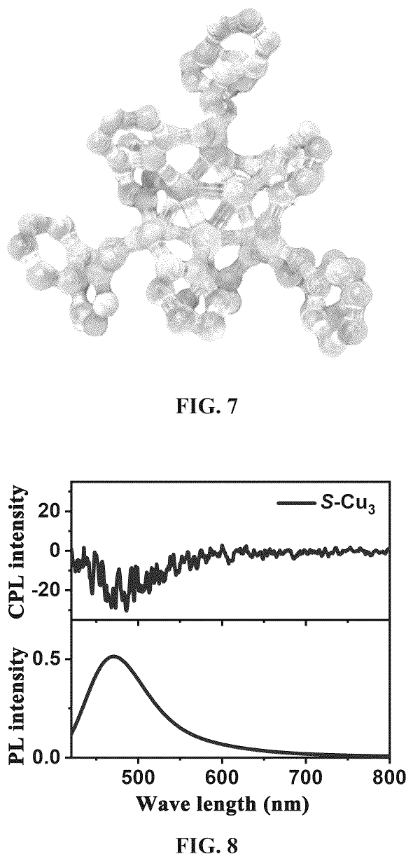

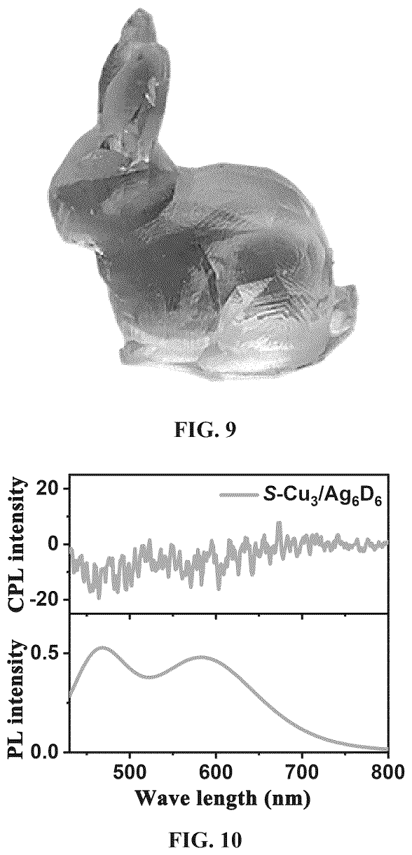

### DETAILED DESCRIPTION

To further illustrate the disclosure, embodiments detailing a chiral N-heterocyclic carbene ligand-protected copper cluster are described below. It should be noted that the following embodiments are intended to describe and not to limit the disclosure.

### Example 1 Synthesis of Chiral Carbene Ligands

A mixture of (1R,2R)-(+)-1,2-diphenyl-1,2-ethanediamine or (1S,2S)-(−)-1,2-diphenyl-1,2-ethanediamine (3.00 g, 14.11 mmol) and glyoxalate monohydrate (1.50 g, 16.50 mmol) in methanol (100 mL) solution was stirred for 4 hours at room temperature. N-bromosuccinimide (NBS) (3.30 g, 18.00 mmol) was added to the mixture and stirred at room temperature overnight. The reaction was quenched by the addition of saturated sodium metabisulfite and the solvent was removed in vacuum to obtain a solid, which was extracted with ethyl acetate by the addition of 5% NaOH solution by mass. The organic layer was dried with anhydrous magnesium sulfate and the solvent was removed in vacuum to obtain chiral imidazole. The synthesized chiral imidazole (2.22 g, 10 mmol) was dissolved in ethanol (100 mL), and 2-(chloromethyl)pyridine hydrochloride (3.35 g, 20.40 mmol) and sodium bicarbonate (2.52 g, 31.12 mmol) were added and refluxed for two days. After filtration, the solvent was removed in vacuum, and a solid was collected, dissolved in dichloromethane, dried with anhydrous magnesium sulfate, and filtered. The solvent was removed in vacuum to obtain an oily product which was mixed with 15 mL of tetrahydrofuran to obtain a powder. The powder was further washed with tetrahydrofuran and dried in vacuum to obtain a white powder. In a flask, the white powder was dissolved in methanol and excess aqueous ammonium hexafluorophosphate solution was dropped, whereby a precipitate was produced immediately. The precipitate was filtered, washed thoroughly with water/methanol and dried in vacuum to obtain the above R/S—NHC^{py}-H·PF₆ chiral carbene ligand.

### Example 2 Synthesis of Chiral Copper Cluster

The obtained chiral ligand R/S—NHC^{py}-H·PF₆ (0.5 mmol, 275 mg) was stirred with excess copper powder (1.5 mmol, 96 mg) in acetonitrile for 24 hours. The suspension was filtered and the volume was reduced to 2 mL, and 50 mL of ether was added to obtain a white crude powdery product. The white crude powdery product was dissolved in 1 mL of acetonitrile, and colorless crystals were obtained through gas diffusion with ether, filtered, washed with ether and dried at room temperature, to yield a chiral copper cluster.

The chiral copper cluster material produced in Example 2 was further characterized as follows.

### Crystal Structure Determination

The X-ray single-crystal diffraction data of the complex were determined on a Rigaku XtaLAB Pro single-crystal diffractometer using single-crystal samples of appropriate size. The data were collected using graphite monochromatized CuKα rays (λ=1.54184 Å) as the diffraction source by @-scanning at 200 K temperature, corrected through Lp factor and semi-empirical absorption. Structural analysis comprised acquiring an initial structure by a direct method with the SHELXS-97 program, and then the initial structure was refined by the full-matrix least-squares method using the SHELXL-97 program. All non-hydrogen atoms were refined using an anisotropic thermal parameter method. All hydrogen atoms were refined using an isotropic thermal parameter method. Detailed crystallographic data are shown in Table 1.

TABLE 1
Main crystallographic data of copper
cluster material of the disclosure

| Compound | R—Cu₃ | S—Cu₃ |
|---|---|---|
| CCDC number | 2251671 | 2251672 |
| Empirical formula | C₁₇₇H_{166.5}Cu₆F₃₆N_{31.5}P₆ | C₁₇₇H_{166.5}Cu₆F₃₆N_{31.5}P₆ |
| Formula weight | 3985.97 | 3985.97 |
| Temperature/K | 200.00(10) | 200.00(10) |
| Crystal system | monoclinic | monoclinic |
| Space group | P2₁ | P2₁ |
| a/Å | 13.5254(2) | 13.5669(2) |
| b/Å | 51.4781(5) | 51.5432(6) |
| c/Å | 14.9912(2) | 15.0017(2) |
| α/° | 90 | 90 |
| β/° | 116.3120(10) | 116.211(2) |
| γ/° | 90 | 90 |
| Volume/Å³ | 9356.4(2) | 9411.7(3) |
| Z | 2 | 2 |
| ρ_{calc} g/cm³ | 1.451 | 1.407 |
| μ/mm⁻¹ | 2.041 | 2.029 |
| F(000) | 4074.0 | 4074.0 |
| Crystal size/mm³ | 0.08 × 0.06 × 0.03 | 0.08 × 0.05 × 0.03 |
| Radiation | CuKα (λ = 1.54184) | CuKα (λ = 1.54184) |
| 2Θ range for data | 6.578 to 148.96 | 6.788 to 146.422 |
| collection/° | | |
| Index ranges | −16 ≤ h ≤ 16, −63 ≤ | −14 ≤ h ≤ 16, −63 ≤ |
| | k ≤ 64, −12 ≤ l ≤18 | k ≤ 63, −18 ≤ l ≤ 9 |
| Reflections | 98925 | 93573 |
| collected | | |
| dependent | 34299 (Rᵢₙₜ = 0.0397, | 34576 (Rᵢₙₜ = 0.0342, |
| reflections | R_{sigma} = 0.0435) | R_{sigma} = 0.0363) |
| Data/restraints/ | 34299/353/2441 | 34576/307/2415 |
| parameters | | |
| Goodness-of-fit on | 1.074 | 1.058 |
| F² | | |
| Final R indexes | R₁ = 0.0726, | R₁ = 0.0732, |
| (I >= 2σ (I)) | wR₂ = 0.1988 | wR₂ = 0.2023 |
| Final R indexes (all | R₁ = 0.0789, | R₁ = 0.0759, |
| data) | wR₂ = 0.2036 | wR₂ = 0.2053 |
| Largest diff. peak/ | 1.31/−0.62 | 1.41/−0.79 |
| hole/e Å⁻³ | | |
| Flack parameters | 0.008(8) | 0.027(8) |

R₁ = Σ||Fₒ| − |F_{c}|Σ/||Fₒ|.
wR₂ = (Σw(Fₒ² − F_{c}²)²/Σw(Fₒ²)²)^{1/2}.

It will be obvious to those skilled in the art that changes and modifications may be made, and therefore, the aim in the appended claims is to cover all such changes and modifications.

What is claimed is:

1. A chiral N-heterocyclic carbene ligand-protected copper cluster, having a formula of C₁₇₇H_{166.5}Cu₆F₃₆N_{31.5}P₆, abbreviated as R/S—Cu₃, belonging to a monoclinic crystal system and having a chiral space group P2₁, R—Cu₃: a=13.5254(2) Å, b=51.4781(5) Å, c=14.9912(2) Å, α=90°, β=116.3120(10), γ=90°, V=9356.4(2) Å³; S—Cu₃: a=13.5669(2) Å, b=51.5432(6) Å, c=15.0017(2) Å, α=90°, β=116.211(2), γ=90°, V=9411.7(3) Å³;
the chiral N-heterocyclic carbene ligand having a formula as follows:

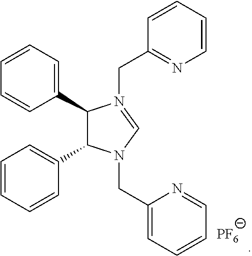

2. The copper cluster of claim 1, comprising three copper metal atoms, three organic ligands and three free hexafluorophosphate anions in a periphery of the three copper metal atoms; wherein the three copper metal atoms form a regular triangular framework having a flat surface; imidazole C atoms of chiral N-heterocyclic carbene ligand and a Cu₃ backbone surface form a coplanar C—Cu—C—Cu—C—Cu six-membered ring; the six-membered ring comprises three C—Cu—Cu isosceles triangles, which share one edge with the regular triangular framework of Cu₃; and each Cu atom is coordinated with two pyridines of two adjacent N-heterocyclic carbene ligands.
